(19) United States
(12) Patent Application Publication
(10) Pub. No.: US 2026/0316063 A1
(43) Pub. Date: Oct. 15, 2026

(54) ZEOLITE COMPOSITE CATALYSTS AND METHODS USING THE SAME

(71) Applicant: Saudi Arabian Oil Company, Dhahran (SA)

(72) Inventors: Veera Venkata Ramakrishna Tammana, Dhahran (SA); Ahmad A. Jazzar, Dhahran (SA); Hashim Nabeel M. Al Khunaizi, Quatif (SA)

(73) Assignee: Saudi Arabian Oil Company, Dhahran (SA)

(21) Appl. No.: 19/179,141

(22) Filed: Apr. 15, 2025

(51) Int. Cl. B01J 29/48; B01J 29/22; B01J 29/24; B01J 29/26; B01J 29/44; B01J 29/46; B01J 29/80; B01J 35/63; B01J 35/64; B01J 37/02; B01J 37/08; B01J 37/12; C07C 6/12

(52) U.S. Cl. CPC B01J 29/48; B01J 29/22; B01J 29/24; B01J 29/26; B01J 29/44; B01J 29/46; B01J 29/80; B01J 35/63; B01J 35/647; B01J 37/0201; B01J 37/08; B01J 37/12; C07C 6/126; B01J 2229/18; B01J 2229/40; C07C 2529/22; C07C 2529/24; C07C 2529/26; C07C 2529/44; C07C 2529/46; C07C 2529/48; C07C 2601/16

(57) ABSTRACT

A method of producing a zeolite composite catalyst may comprise: impregnating a zeolite composite with molybdenum to form a molybdenum impregnated zeolite composite, wherein the zeolite composite comprises a mesoporous mordenite and a mesoporous ZSM-5; calcining the molybdenum impregnated zeolite composite to form a calcined, molybdenum impregnated zeolite composite; impregnating the calcined, molybdenum impregnated zeolite composite with one or more active metals selected from platinum, ruthenium, nickel, tungsten, chromium, cerium, or gold, to yield a multi-metallic impregnated zeolite composite; and calcining the multi-metallic impregnated zeolite composite to produce the zeolite composite catalyst.

### TECHNICAL FIELD

Embodiments of the present disclosure generally relate to chemical processing and, more specifically, to catalysts and uses thereof.

### TECHNICAL BACKGROUND

Aromatic hydrocarbon compounds derived from petrochemical sources (e.g., benzene (C₆H₆), toluene (methylbenzene, C₇H₈), and xylenes (dimethylbenzenes, C₈H₁₀ isomers)) may be used as starting materials for a wide range of consumer products. Xylenes in particular include three isomers of dimethylbenzene, namely: 1,2-dimethylbenzene (orthoxylene or o-xylene), 1,3-dimethylbenzene (meta-xylene or m-xylene), and 1,4-dimethylbenzene (para-xylene or p-xylene). The three isomers of xylene may be used in the synthesis of a number of useful products, creating a strong demand for xylenes.

Xylene may be produced from heavy reformate. Typically, heavy reformate contains 90 weight (wt.) % to 95 wt. % C₉ and 5 wt. % to 10 wt. % C₁₀ aromatic compounds. Among the C₉ components, trimethylbenzenes (TMBs) (50 wt. % to 60 wt. %) and methylethylbenzenes (MEBs) (30 wt. % to 40 wt. %) are the major constituents. One of the economically viable options is to convert the heavy aromatics in the heavy reformate into valuable products, such as xylenes via a transalkylation process. Specifically, heavy reformate can be subjected to transalkylation either alone or with toluene (C₇) for the production of xylenes (C₈) and benzene (C₆). Because many different compounds may be present in the heavy reformate, multiple parallel and consecutive reactions may take place.

These conventional means to produce xylenes from reformate result in excessive yields of the byproduct ethylbenzene. Ethylbenzene can be difficult to separate from the xylene, reducing the quality and value of the xylenes produced.

### BRIEF SUMMARY

Described herein are zeolite composite catalysts, and methods for making such, that comprise mesoporous ZSM-5 and mesoporous mordenite, impregnated with molybdenum and one or more active metals. In some embodiments, the zeolite composite catalyst is formed by impregnating a zeolite composite comprising the mesoporous ZSM-5 and mesoporous mordenite with molybdenum, calcining the product, then, impregnating it with one or more active metals, and then calcining the product again. In one or more embodiments, such catalyst may be utilized for converting heavy reformates to produce xylenes with a relatively small amount of ethylbenzene. It is believed that, according to some embodiments, the use of a catalyst having both the molybdenum and the one or more active metals for the conversion of heavy reformates may result in both increased overall conversion rates and reduced yields of ethylbenzene, relative to catalysts comprising only one metal. Surprisingly, it has been presently discovered that the use of a catalyst where the molybdenum and the one or more active metals are impregnated in separate impregnation steps, separated by a calcination step, results in further reduced yields of ethylbenzene, relative to catalysts where the molybdenum and the one or more active metals are both impregnated into the zeolite composite in a single step.

According to one embodiment, a method of producing a zeolite composite catalyst, may comprise: impregnating a zeolite composite with molybdenum to form a molybdenum impregnated zeolite composite, wherein the zeolite composite comprises a mesoporous mordenite and a mesoporous ZSM-5; calcining the molybdenum impregnated zeolite composite to form a calcined, molybdenum impregnated zeolite composite; impregnating the calcined, molybdenum impregnated zeolite composite with one or more active metals selected from platinum, ruthenium, nickel, tungsten, chromium, cerium, or gold, to yield a multi-metallic impregnated zeolite composite; and calcining the multi-metallic impregnated zeolite composite to produce the zeolite composite catalyst.

These and other embodiments are described in more detail in the Detailed Description. It is to be understood that both the foregoing general description and the following detailed description present embodiments of the presently disclosed technology, and are intended to provide an overview or framework for understanding the nature and character of the technology as it is claimed. The accompanying drawings are included to provide a further understanding of the presently disclosed technology and are incorporated into and constitute a part of this specification. The drawings illustrate various embodiments and, together with the description, serve to explain the principles and operations of the presently disclosed technology. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 is a flow chart depicting the synthesis of a zeolite composite catalyst in accordance with one or more embodiments of the present disclosure.

FIG. 2 is a schematic illustration depicting the conversion of heavy reformate into xylenes in accordance with one or more embodiments of the present disclosure.

For the purpose of describing the simplified schematic illustrations and descriptions of the relevant figures, the numerous valves, temperature sensors, electronic controllers and the like that may be employed and well known to those of ordinary skill in the art of certain chemical processing operations are not included. Further, accompanying components that are often included in typical chemical processing operations, such as air supplies, catalyst hoppers, and flue gas handling systems, are not depicted. However, operational components, such as those described in the present disclosure, may be added to the embodiments described in this disclosure.

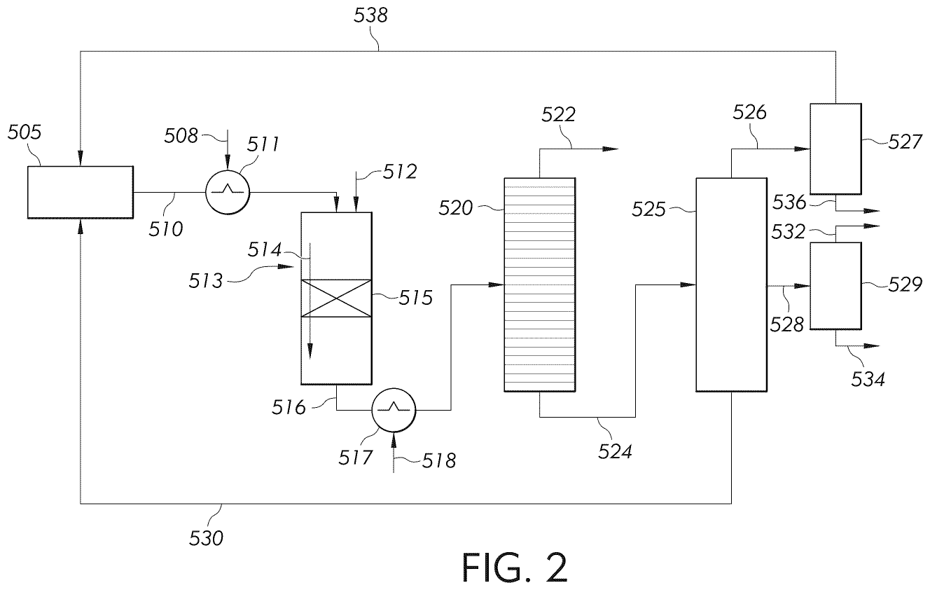

It should further be noted that arrows in FIG. 2 refer to process streams. However, the arrows may equivalently refer to transfer lines which may serve to transfer process streams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows which do not connect two or more system components signify a product stream which exits the depicted system or a system inlet stream which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a system product.

Additionally, arrows in FIG. 2 may schematically depict process steps of transporting a stream from one system component to another system component. For example, an arrow from one system component pointing to another system component may represent “passing” a system component effluent to another system component, which may include the contents of a process stream “exiting” or being “removed” from one system component and “introducing” the contents of that product stream to another system component. It should be understood that arrows in the relevant figures are not indicative of necessary or essential steps.

It should be understood that according to the embodiments presented in FIG. 2, an arrow between two system components may signify that the stream is not processed between the two system components. In other embodiments, the stream signified by the arrow may have substantially the same composition throughout its transport between the two system components. Additionally, it should be understood that in one or more embodiments, an arrow may represent that at least 75 wt. %, at least 90 wt. %, at least 95 wt. %, at least 99 wt. %, at least 99.9 wt. %, or even 100 wt. % of the stream is transported between the system components. As such, in some embodiments, less than all of the streams signified by an arrow may be transported between the system components, such as if a slip stream is present.

It should be understood that a stream may be named for a major component. Such a stream may comprise a majority of the named component, such as at least 50 wt. %, at least 60 wt. %, at least 70 wt. %, at least 80 wt. %, at least 90 wt. %, at least 95 wt. %, at least 97 wt. %, at least 99 wt. %, at least 99.9 wt. %, at least 99.99 wt. %, or even 100 wt. % of the named component, on the basis of the total weight of the respective stream.

It should be understood that two or more process streams are “mixed” or “combined” when two or more lines intersect in the schematic flow diagrams of the relevant figures. Mixing or combining may also include mixing by directly introducing both streams into a like reactor, separation device, or other system component. For example, it should be understood that when two streams are depicted as being combined directly prior to entering a separation unit or reactor, that in some embodiments the streams could equivalently be introduced into the separation unit or reactor and be mixed in the reactor.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Reference will now be made in detail to embodiments of the method of making a zeolite composite catalyst. Generally, the concepts described with respect to one figure can be equally applicable in another, as would be understood by those skilled in the art.

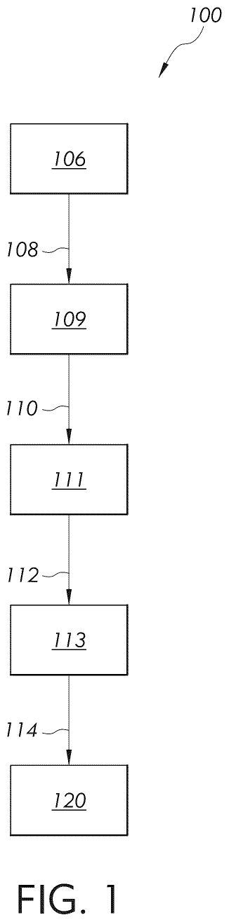

Referring first to FIG. 1, a method 100 of producing a zeolite composite catalyst 120 may comprise an impregnating step 108, where a zeolite composite 106 may be impregnated with molybdenum to form a molybdenum impregnated zeolite composite 109, a calcining step 110 where the molybdenum impregnated zeolite composite 109 is calcined to form a calcined, molybdenum impregnated zeolite composite 111, an impregnating step 112 where the calcined, molybdenum impregnated zeolite composite 111 may be impregnated with one or more active metals selected from platinum, ruthenium, nickel, tungsten, chromium, cerium, or gold, to yield a multi-metallic impregnated zeolite composite 113, and a second calcining step 114 where the multi-metallic impregnated zeolite composite 113 is calcined to produce the zeolite composite catalyst 120.

As used herein, a zeolite composite 106 refers to a mixture comprising two or more zeolites, such as a mixture of the mesoporous mordenite and the mesoporous ZSM-5. In some embodiments, the zeolite composite 106 may further comprise additional components, such as a binder. The zeolite composite 106 may be mixed by co-extrusion. In some embodiments, the zeolite composite 106 may be a solid material, such as an extrudate. In some embodiments, the zeolite composite 106 may be a particle (or plurality of particles).

In some embodiments, the zeolite composite 106 may comprise a mesoporous mordenite and a mesoporous ZSM-5. In some embodiments, the zeolite composite 106 may be formed by mixing the mesoporous mordenite and the mesoporous ZSM-5, and optionally, also with a binder. Methods for producing mesoporous mordenite and mesoporous ZSM-5, according to one or more embodiments, are described later herein.

The mesoporous mordenite may comprise mordenite which has a mesoporous pore structure. In some embodiments, the mordenite may have been modified to have a mesoporous pore structure. Mordenite is a zeolite mineral having the formula Ca, Na₂, K₂, Al₂Si₁₀O₂₄·7H₂O. Mordenite's molecular framework includes chains of five-membered rings of linked silicate and aluminate tetrahedra (four oxygen atoms arranged at the points of a triangular pyramid about a central silicon or aluminum atom). The mordenite may be synthetic mordenite or naturally mined mordenite. Ordinarily, mordenite is microporous and lacks mesoporosity. However, the present mesoporous mordenite has mesopores as well. In some embodiments, a microporous mordenite has been modified to form the mesoporous mordenite. For example, the mordenite may have been modified by the surfactant assisted process described below.

The mesoporous mordenite may comprise mesopores, or both mesopores and micropores. Mesopores are defined as pores having a diameter of from 2 nm to 50 nm. In some embodiments, the mesopores may have pore diameters of from 4 nm to 50 nm, from 6 nm to 50 nm, from 8 nm to 50 nm or even from 10 nm to 50 nm. Micropores are defined as pores having a diameter of less than 2 nm.

The mesoporous mordenite may have a mesopore volume of at least 0.2 cm³/g, such as greater than 0.5 cm³/g, greater than 1.0 cm³/g, greater than 1.5 cm³/g, from 0.2 cm³/g to 3.0 cm³/g, from 0.2 cm³/g to 0.4 cm³/g, from 0.4 cm³/g to 0.6 cm³/g, from 0.6 cm³/g to 1.0 cm³/g, from 1.0 cm³/g to 1.4 cm³/g, from 1.4 cm³/g to 1.8 cm³/g, from 1.8 cm³/g to 2.2 cm³/g, from 2.2·cm³/g to 2.6 cm³/g, from 2.6 cm³/g to 3.0 cm³/g, or any combination of two or more of these ranges. Mesopore volume refers to the volume of mesopores in the material. Mesopore volume may be determined by the well-known Brunauer-Emmett-Teller (BET) method using nitrogen adsorption. Generally, the use of a mesoporous mordenite having a relatively high mesopore volume, such as greater than 0.2 cm³/g, may result in improved ability to process heavy feeds as the large aromatic molecules in the feed may be better able to access the pores.

The mesoporous mordenite may also have a micropore volume of from 0.05 cm³/g to 3.0 cm³/g, such as from 0.05 cm³/g to 0.1 cm³/g, from 0.1 cm³/g to 0.15 cm³/g, from 0.15 cm³/g to 0.2 cm³/g, from 0.2 cm³/g to 0.4 cm³/g, from 0.4 cm³/g to 0.6 cm³/g, from 0.6 cm³/g to 1.0 cm³/g, from 1.0 cm³/g to 1.4 cm³/g, from 1.4 cm³/g to 1.8 cm³/g, from 1.8 cm³/g to 2.2 cm³/g, from 2.2·cm³/g to 2.6 cm³/g, from 2.6 cm³/g to 3.0 cm³/g, or any combination of two or more of these ranges. Micropore volume refers to the volume of micropores in the material. Micropore volume may be determined by the well-known Brunauer-Emmett-Teller (BET) method using nitrogen adsorption. Without being limited by theory, the volume of micropores at least partially may dictate the nature of functionality of the particular zeolite. For example, the micropore volume may help control the activity and/or selectivity of the selectivity of the resulting catalyst.

In one or more embodiments, the mesoporous mordenite may have a total pore volume of at least 0.20 cm³/g, such as from 0.20 to 3.0 cm³/g, from 0.2 to 0.5 m³/g, from 0.5 to 1.0 m³/g, from 1.0 to 1.5 m³/g, from 1.5 to 2.0 m³/g, from 2.0 to 2.5 m³/g, from 2.5 to 3.0 m³/g, or any combination of two or more of these ranges. In some embodiments, the mesoporous mordenite may have an average pore diameter of at least 20 angstroms (2 nanometers (nm)), such as at least 3 nm, from 2 nm to 10 nm, or from 3 nm to 10 nm. Total pore volume may be determined by the well-known Brunauer-Emmett-Teller (BET) method using nitrogen adsorption. Without being limited by theory, the total pore volume may at least partially control the nature and functionality of the zeolite. For example, total pore volume may help to control the ability of the mordenite to process large aromatic molecules in the feed and/or may help control the activity and/or selectivity of the resulting catalyst.

The mesoporous mordenite may have a surface area defined by a Brunauer-Emmett-Teller (BET) analysis (also known as the Specific Surface Area) of at least 300 meters squared per gram (m²/g), such as at least 400 m²/g, at least 450 m²/g, or at least 500 m²/g. The BET surface area represents the total surface area of a material per unit of mass.

The mesoporous mordenite may have a SiO₂/Al₂O₃ molar ratio of 20 to 300, such as from 20 to 40, from 40 to 60, from 60 to 80, from 80 to 100, from 100 to 120, from 120 to 140, from 140 to 160, from 160 to 180, from 180 to 200, from 200 to 220, from 220 to 240, from 240 to 260, from 260 to 280, from 20 to 300, or any combination of two or more of these ranges. Without being limited by theory, it is believed that the SiO₂/Al₂O₃ molar ratio may help control the crystal structure and may be at least partially responsible for the selectivity of the resulting catalyst for the desired product.

Still referring to FIG. 1, the mesoporous ZSM-5 that is included in the zeolite composite 106 may comprise ZSM-5 zeolite which has a mesoporous pore structure, such as ZSM-5 zeolite that has been modified to be mesoporous. ZSM-5 is an aluminosilicate zeolite of the pentasil family. In ZSM-5, several pentasil units are linked together by oxygen bridges to form pentasil chains. A pentasil unit has eight five-membered rings. Ordinarily, ZSM-5 is microporous and lacks mesoporosity. However, present mesoporous ZSM-5 has mesopores, or both micropores and mesopores. In some embodiments, a microporous ZSM-5 may have been modified to form the mesoporous ZSM-5. For example, the microporous ZSM-5 may have been modified by the surfactant assisted process described below.

The mesoporous ZSM-5 may have a mesopore volume of at least 0.1 cm³/g, such as at least 0.15 cm³/g, at least 0.2 cm³/g, from 0.1 cm³/g to 0.15 cm³/g, from 0.15 cm³/g to 0.2 cm³/g, from 0.2 cm³/g to 3.0 cm³/g, from 0.1 cm³/g to 0.15 cm³/g, from 0.15 cm³/g to 0.2 cm³/g, from 0.2 cm³/g to 0.4 cm³/g, from 0.4 cm³/g to 0.8 cm³/g, from 0.8 cm³/g to 1.0 cm³/g, from 1.0 cm³/g to 1.4 cm³/g, from 1.4 cm³/g to 1.8 cm³/g, from 1.8 cm³/g to 2.2 cm³/g, from 2.2·cm³/g to 2.6 cm³/g, from 2.6 cm³/g to 3.0 cm³/g, or any combination of two or more of these ranges. Generally, the use of a mesoporous ZSM-5 having a relatively high mesopore volume, such as greater than 0.2 cm³/g may result in improved ability to process heavy feeds as the large aromatic molecules in the feed may be better able to access the pores.

The mesoporous ZSM-5 may also have a micropore volume of at least 0.1 cm³/g, at least 0.15 cm³/g, from 0.1 cm³/g to 0.15 cm³/g, from 0.15 cm³/g to 0.2 cm³/g, from 0.2 cm³/g to 3.0 cm³/g, such as from 0.2 cm³/g to 0.4 cm³/g, from 0.4 cm³/g to 0.8 cm³/g, from 0.8 cm³/g to 1.0 cm³/g, from 1.0 cm³/g to 1.4 cm³/g, from 1.4 cm³/g to 1.8 cm³/g, from 1.8 cm³/g to 2.2 cm³/g, from 2.2·cm³/g to 2.6 cm³/g, from 2.6 cm³/g to 3.0 cm³/g, or any combination of two or more of these ranges. Without being limited by theory, the volume of micropores at least partially may dictate the nature of functionality of the particular zeolite. For example, the micropore volume may help control the activity and/or selectivity of the resulting catalyst.

In one or more embodiments, the mesoporous ZSM-5 may have a total pore volume of 0.20 to 3.0 cubic centimeters per gram (cm³/g), such as from 0.2 to 0.5 m³/g, from 0.5 to 1.0 m³/g, from 1.0 to 1.5 m³/g, from 1.5 to 2.0 m³/g, from 2.0 to 2.5 m³/g, from 2.5 to 3.0 m³/g, or any combination of two or more of these ranges. In some embodiments, the mesoporous ZSM-5 may have an average pore diameter of at least 20 angstroms (2 nanometers (nm)), such as at least 3 nm, from 2 nm to 10 nm, or from 3 nm to 10 nm. Without being limited by theory, the total pore volume may at least partially control the nature and functionality of the zeolite. For example, total pore volume may help to control the ability of the resulting catalyst to process large aromatic molecules in the feed.

The mesoporous ZSM-5 may have a BET surface area of at least 300 m²/g, such as at least 350 m²/g, at least 400 m²/g, at least 450 m²/g, or at least 500 m²/g.

In embodiments, the mesoporous ZSM-5 may have a Si/Al molar ratio of from 5 to 500, such as from 5 to 20, from 20 to 40, from 40 to 60, from 60 to 80, from 80 to 100, from 100 to 150, from 150 to 200, from 200 to 250, from 250 to 300, from 300 to 350, from 350 to 400, from 400 to 450, from 450 to 500, or any combination of two or more of these ranges.

A ratio of the mesoporous mordenite to mesoporous ZSM-5 may be from 1:1 to 5:1 by weight. For example, the weight ratio of the mesoporous mordenite to the mesoporous ZSM-5 may be from 1:1 to 2:1, from 2:1 to 3:1, from 3:1 to 4:1, from 4:1 to 5:1, or any combination of two or more of these ranges.

Still referring to FIG. 1, the mesoporous mordenite and mesoporous ZSM-5 may be mixed to form the zeolite composite 106. The mesoporous ZSM-5 and the mesoporous mordenite may be mixed with a binder to form the zeolite composite 106. A refractory binder or matrix may be utilized to facilitate fabrication of the catalyst, to provide strength, and to reduce fabrication costs. Suitable binders may include inorganic oxides, such as one or more of alumina, magnesia, zirconia, chromia, titania, boric, phosphate, zinc oxide and silica. In one embodiment, the binder is an alumina based binder. One commercial embodiment of the alumina binder is Cataloid AP-3, commercially available from Catalysts & Chemicals Industries Co., Ltd (CCIC), Japan. The zeolites may be mixed in dry powdered form with the alumina binder in aqueous form to yield a homogeneous mixture, thus ensuring homogeneous composition of the extrudates formed. In one or more embodiments, the ratio by weight of solid zeolite composite to binder may be from 4:1 (80 wt. % zeolite and 20 wt. % binder) to 2:1, such as from 4:1 to 3:1 or from 3:1 to 2:1.

According to one or more embodiments, mixing the mesoporous mordenite and mesoporous ZSM-5 to form the zeolite composite 106 may comprise physically mixing the mesoporous mordenite and mesoporous ZSM-5, and binder. Suitable physical mixing methods may include any sufficiently high shear mixing method, such as co-extrusion. The mixing (e.g., extrusion) may be conducted at a temperature of 40° C. to 150° C. for a duration of 30 minutes to 2 hours.

As mentioned herein, the method 100 may comprise impregnating the molybdenum and the one or more active metals into the zeolite composite 106 in separate steps 108, 112. In some embodiments, the use of separate molybdenum impregnation in step 108 and one or more active metals impregnation in step 112 may result in further decreased ethylbenzene production while maintaining catalyst activity and xylene production. Without being limited by theory, this improvement is believed to be due to differential evaporation rates of different metal salts in the zeolite-binder matrix during drying. These differential evaporation rates may lead to uneven dispersal of the metals in the matrix when done in co-addition mode (e.g., both metals are included in a single impregnation solutions. However, when performed in a sequential addition mode (e.g., the method 100 of FIG. 1), this problem may be avoided.

Still referring to FIG. 1, the zeolite composite 106 may be subjected to a molybdenum impregnation step 108, a first calcining step 110, impregnation of one or more active metals in step 112, and a second calcining step 114, thereby producing the zeolite composite catalyst 120.

In the molybdenum impregnation step 108, the zeolite composite may be impregnated with molybdenum to form a molybdenum impregnated zeolite composite 109. The molybdenum impregnation in step 108 may be by wet impregnation or incipient wetness impregnation, co-mulling, adding metal to zeolite phase or binding phase exclusively before extrusion process. In some embodiments, the zeolite composite 106 may be placed in a solution comprising molybdenum precursors. The molybdenum precursors may adhere to the zeolite composite 106. Suitable molybdenum precursors may include, for example, ammonium molybdate, molybdenum chloride and molybdophosphoric acid. In some embodiments, the solution may comprise at least 90 wt. %, at least 95 wt. %, at least 98 wt. %, at least 99 wt. %, at least 99.9 wt. %, or at least 99.99 wt. % of the combined weight of the molybdenum precursors and water, on the basis of the total weight of the solution.

The molybdenum impregnated zeolite composite 109 may then be subjected to a first calcining step 110 to form a calcined molybdenum impregnated zeolite composite. In first calcining step 110, the molybdenum impregnated zeolite composite 109 may be heated for 4 to 8 hours at a temperature of 400 to 600° C., such as from 4 hours to 5 hours, from 5 hours to 6 hours from 6 hours to 7 hours, from 7 hours to 8 hours, or any combination of two or more of these ranges, and a temperature of from 400° C. to 425° C., from 425° C. to 450° C., from 450° C. to 475° C., from 475° C. to 500° C., from 500° C. to 525° C., from 525° C. to 550° C., from 550° C. to 575° C., from 575° C. to 600° C., or any combination of two or more of these ranges. The first calcining step 110 may take place in an oxidizing atmosphere, such as air.

The calcined, molybdenum impregnated zeolite composite 111 may then be subjected to a one or more active metals impregnations in step 112 to form a multi-metallic impregnated zeolite composite. The impregnation may be by wet impregnation or incipient wetness impregnation. In step 112, the calcined, molybdenum impregnated zeolite composite 111 may be placed in a solution comprising precursors of the one or more active metals and the precursors may adhere to the calcined, molybdenum impregnated zeolite composite 111. The one or more active metals may be selected from platinum, ruthenium, nickel, tungsten, chromium, cerium, or gold, to yield a multi-metallic impregnated zeolite composite. In some embodiments, the one or more active metals may be selected from platinum, ruthenium, and nickel. Suitable platinum precursors may include, without limitation, Pt(NH₃)Cl₂, PtCl₂, PtCl₄, (NH₄)₂PtCl₆, (NH₄)₂Pt(NO₃)₂, Na₂PtCl₆·6H2O, H₂PtCl₆·6H2O, Na₂PtCl₄·4H₂O, and (NH₄)₂PtCl₄. Suitable nickels precursors may include, without limitation, nickel chloride, nickel nitrate, and nickel sulfate. Suitable ruthenium precursors may include, without limitation, ruthenium chloride. Suitable tungsten precursors may include ammonium meta tungstate hydrate. Suitable chromium precursors may include chromium (III) nitrate and chromium hexacarbonyl. Suitable cerium precursors may include cerium (III) nitrate hexahydrate. Suitable gold precursors may include gold (III) chloride hexahydrate. In some embodiments, the solution may comprise at least 90 wt. %, at least 95 wt. %, at least 98 wt. %, at least 99 wt. %, at least 99.9 wt. %, or at least 99.99 wt. % of the combined weight of the water and the one or more active metals precursors, on the basis of the total weight of the solution. Optionally, the multi-metallic impregnated zeolite composite 113 may be dried after impregnation. In one embodiment, the drying occurs for at least 2 hours at from 80° C. to 120° C.

The multi-metallic impregnated zeolite composite 113 may then be subjected to second calcining step 114, thereby forming zeolite composite catalyst 120. In second calcining step 114, the multi-metallic impregnated zeolite composite 113 may be heated for 4 to 8 hours at a temperature of 400 to 600° C., such as from 4 hours to 5 hours, from 5 hours to 6 hours from 6 hours to 7 hours, from 7 hours to 8 hours, or any combination of two or more of these ranges, and a temperature of from 400° C. to 425° C., from 425° C. to 450° C., from 450° C. to 475° C., from 475° C. to 500° C., from 500° C. to 525° C., from 525° C. to 550° C., from 550° C. to 575° C., from 575° C. to 600° C., or any combination of two or more of these ranges. The second calcining step 114 may take place in an oxidizing atmosphere, such as air.

Now described herein is one suitable method of forming a mesoporous mordenite. In some embodiments, the mesoporous mordenite may be formed by a surfactant assisted modification method. The surfactant assisted modification method may comprise forming the mesoporous mordenite by: dissolving a microporous mordenite in a first alkaline solution to yield a dissolved mordenite solution. Then, condensing the dissolved mordenite solution by adjusting a pH of the dissolved mordenite solution and aging the pH adjusted dissolved mordenite solution to yield a solid mordenite. Finally, the surfactant assisted modification method may comprise heating the solid mordenite to form the mesoporous mordenite.

The starting material for the surfactant assisted modification method may be a microporous mordenite. The microporous mordenite is a mordenite zeolite material have a microporous pore structure. The microporous mordenite may have a mesopore volume less than 3.0 cm³/g, such as less than 2.5 cm³/g, less than 2.0 cm³/g, less than 1.0 cm³/g, less than 0.5 cm³/g, less than 0.2 cm³/g, or even less than 0.1 cm³/g.

The microporous mordenite may have a Si/Al molar ratio of 20 to 300, such as from 20 to 40, from 40 to 60, from 60 to 80, from 80 to 100, from 100 to 120, from 120 to 140, from 140 to 160, from 160 to 180, from 180 to 200, from 200 to 220, from 220 to 240, from 240 to 260, from 260 to 280, from 20 to 300, or any combination of two or more of these ranges. Without being limited by theory, it is believed that the Si/Al molar ratio may help control the crystal structure and may be at least partially responsible for the selectivity of the catalyst for the desired product.

In the dissolving step, the microporous mordenite may be placed in a first alkaline solution comprising a surfactant. Generally, the surfactant may also be referred to as a templating agent for the zeolite catalyst. In embodiments, surfactant may be a cationic surfactant. The cationic surfactant may include a quaternary ammonium compound. One example of suitable quaternary ammonium cationic surfactant may include cetyltrimethyl ammonium bromide (CTAB). Various amounts of surfactant are contemplated for inclusion in the catalyst precursor. For example, the catalyst precursor may include 1 wt. % to 10 wt. % surfactant, for example CTAB, such as from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, from 2 wt. % to 8 wt. %, from 3 wt. % to 6 wt. %, from 4 wt. % to 5 wt. %, or any combination of two or more of these ranges of the surfactant, on the basis of the total weight of the first alkaline solution.

Still describing the fabrication of mesoporous mordenite, the dissolution may occur slowly in the presence of the surfactant by gradual heating for at least 4 hours, such as at least 8 hours, at least 12 hours, at least 16 hours, at least 20 hours, or at least 24 hours. Mesopores may then be generated using a template mediated technique, where a partially dissolved zeolite is thermally recrystallized around a selected template (e.g., the surfactant) which dictates the pore size of the regrown structure. In this way, the unutilized desilicated source is utilized to produce the mesophases. Various heating processes or elements are contemplated. For example, the heating may occur at a temperature of 50° C. to 150° C., or a temperature of 90° C. to 110° C.; and/or a time of from 30 minutes to 48 hours.

Various first alkaline solutions are contemplated. Suitable first alkaline solutions may include those with a pH of from 0.1 to 0.5, such as from 0.1 to 0.2, from 0.2 to 0.3, from 0.3 to 0.4, from 0.4 to 0.5, or any combination of two or more of these ranges. Without being limited by theory, it is believed that the specific pH values described herein may result in more efficient formation of the mesoporous mordenite in the desired structure. In some specific embodiments, the first alkaline solution may be an aqueous solution of 0.3 M to 0.5 M NaOH. The temperature of the first alkaline solution may be from 45° C. to 100° C., such as from 45° C. to 50° C., from 50° C. to 60° C., from 60° C. to 70° C., from 70° C. to 80° C., from 80° C. to 90° C., from 90° C. to 100° C., or any combination of two or more of these ranges. Additionally, the dissolving step 204 may occur under stirring, in order to further speed dissolution.

In one embodiment, the first alkaline solution may comprise NaOH. In specific embodiments, the alkaline solution may comprise 0.01 to 0.6 M NaOH, such as from 0.01 to 0.05 M NaOH, from 0.05 to 0.1 M NaOH, from 0.1 to 0.2 M NaOH, from 0.2 to 0.3 M NaOH, from 0.3 to 0.4 M NaOH, from 0.4 to 0.5 M NaOH, from 0.5 M NaOH to 0.6 M NaOH, or any combination of two or more of these ranges. Without being bound by theory, it is surprisingly discovered that controlling the molarity of the NaOH is a parameter that impacts the mesostructure of the zeolite composite catalyst. It is believed the NaOH is responsible for breaking the chemical structure of the zeolites, thus a larger concentration of the NaOH leads to increased dissolution of the zeolite structure which is recrystallized in the presence of CTAB to form the mesostructure of the zeolite composite catalyst.

The first alkaline solution may comprise at least 95 wt. %, such as at least 98 wt. %, at least 99 wt. %, at least 99.9 wt. %, or at least 99.99 wt. % of the surfactant, the base, and water, on the basis of the total weight of the first alkaline solution.

Once formed, the dissolved mordenite solution may then be condensed to yield a solid mordenite. Condensing the dissolved mordenite solution may comprise adjusting the pH of the dissolved mordenite solution and subsequently, aging the pH adjusted dissolved mordenite solution.

Still referring to the fabrication of the mesoporous mordenite, the surfactant assisted modification method may comprise adjusting the pH of the dissolved mordenite solution. The adjustment of the pH is performed by an acidic solution. Various acids are contemplated. In one embodiment, the acidic solution comprises sulfuric acid. In one specific embodiment, the acid is 2 Normality (N) (equivalents/liter) dilute sulfuric acid. In specific embodiments, the pH is adjusted to less than 10, such as to less than 9, from 8 to 10, from 8 to 9, 9 to 10, 8.5 to 9.5, 8.75 to 9.25, or any combination of two or more of these ranges. Without being limited by theory, it is believed that adjusting the pH to the described range may result in more efficient or complete condensation of the dissolved mordenite solution.

Once the pH is adjusted, the pH adjusted dissolved mordenite solution may be aged. The aging, sometimes referred to as hydrothermal aging, may involve maintaining the pH adjusted dissolved mordenite solution at a temperature of 75 to 125° C. for a duration of 12 to 48 hours. For example, the pH adjusted dissolved zeolite solution may be aged at from 75 to 85° C., from 85 to 95° C., from 95 to 105° C., from 105 to 115° C., from 115 to 125° C., or any combination of two or more of these ranges for a duration of at least 4 hours, such as at least 8 hours, at least 12 hours, at least 16 hours, at least 20 hours, at least 24 hours, from 4 hours to 8 hours, from 8 hours to 12 hours, from 12 hours to 16 hours, from 16 hours to 20 hours, from 20 hours to 24 hours, from 24 hours to 28 hours, from 28 hours to 32 hours, from 32 hours to 36 hours, from 36 hours to 40 hours, from 40 hours to 44 hours, from 44 hours to 48 hours, or any combination of two or more of these ranges. Without being limited by theory, it is believed that during aging, the soluble aluminosilicate species may be hydrothermally condensed to form mesophases. The condensing of the dissolved mordenite solution may be performed under still conditions or agitated conditions. Specifically, under still conditions, the dissolved mordenite solution is not stirred or mixed where conversely under agitated conditions the dissolved mordenite solution is mixed or stirred. The solid products formed from condensing the dissolved mordenite solution may be filtered to form a solid mordenite, washed thoroughly using distilled water, and then dried. For example, the solid zeolite composite may be dried for at least 12 hours at a temperature of from 60° C. to 160° C., such as from 60° C. to 80° C., from 80° C. to 100° C., from 100° C. to 120° C., from 120° C. to 140° C., from 140° C. to 160° C., or any combination of two or more of these ranges.

Following drying, the solid mordenite may then be subjected to ion exchange. The ion exchange may occur in the presence of a nitrate solution, e.g., a solution comprising NH₄NO₃. In one or more embodiments, the solid mordenite is ion exchanged thrice with 0.05M NH₄NO₃ solution at 80° C. for 5 hours.

Then, the solid mordenite may be heated to remove the entrained surfactant. For example, the solid zeolite composite may be heated to 500 to 600° C. for 5 to 8 hours, such as from 500 to 520° C., from 520 to 540° C., from 540 to 560° C., from 560 to 580° C., from 580 to 600° C., or any combination of two or more of these ranges, for from 5 hours to 6 hours, from 6 hours to 7 hours, from 7 hours to 8 hours, or any combination of two or more of these ranges. In some specific embodiments, the solid mordenite may be heated to about 570° C. for 7 hours, or 550° C. for 6 hours to vaporize and remove the surfactant. The heating may occur in the presence of an oxidizing atmosphere, such as air. Thus, the mesoporous mordenite may be formed.

Now described is a method for making mesoporous ZSM-5, where other suitable methods for making mesoporous ZSM-5 are in keeping with the presently disclosed embodiments. In some embodiments, the mesoporous ZSM-5 may be formed by a surfactant assisted modification method. The surfactant assisted modification method may comprise forming the mesoporous ZSM-5 by: dissolving a microporous ZSM-5 in a second alkaline solution to yield a dissolved ZSM-5 solution. Then, condensing the dissolved ZSM-5 solution by adjusting a pH of the dissolved ZSM-5 solution and aging the pH adjusted dissolved ZSM-5 solution to yield a solid ZSM-5. Finally, the surfactant assisted modification method may comprise heating the solid ZSM-5 to form the mesoporous ZSM-5.

The starting material for the surfactant assisted modification method may be a microporous ZSM-5. The microporous ZSM-5 is a ZSM-5 zeolite material having a microporous pore structure. The microporous ZSM-5 may have a mesopore volume less than 3.0 cm³/g, such as less than 2.5 cm³/g, less than 2.0 cm³/g, less than 1.0 cm³/g, less than 0.5 cm³/g, less than 0.2 cm³/g, or even less than 0.1 cm³/g.

In embodiments, the microporous ZSM-5 may have a Si/Al molar ratio of from 5 to 500, such as from 5 to 20, from 20 to 40, from 40 to 60, from 60 to 80, from 80 to 100, from 100 to 150, from 150 to 200, from 200 to 250, from 250 to 300, from 300 to 350, from 350 to 400, from 400 to 450, from 450 to 500, or any combination of two or more of these ranges.

In the dissolving step, the microporous ZSM-5 may be placed in a second alkaline solution comprising a surfactant. Generally, the surfactant may also be referred to as a templating agent for the zeolite catalyst. In embodiments, surfactant may be a cationic surfactant. The cationic surfactant may include a quaternary ammonium compound. One example of a suitable quaternary ammonium cationic surfactant may include cetyltrimethyl ammonium bromide (CTAB). Various amounts of surfactant are contemplated for inclusion in the second alkaline solution. For example, the catalyst precursor may include 1 wt. % to 10 wt. % surfactant, for example CTAB, such as from 1 wt. % to 2 wt. %, from 2 wt. % to 3 wt. %, from 3 wt. % to 4 wt. %, from 4 wt. % to 5 wt. %, from 5 wt. % to 6 wt. %, from 6 wt. % to 7 wt. %, from 7 wt. % to 8 wt. %, from 8 wt. % to 9 wt. %, from 9 wt. % to 10 wt. %, from 2 wt. % to 8 wt. %, from 3 wt. % to 6 wt. %, from 4 wt. % to 5 wt. %, or any combination of two or more of these ranges of the surfactant, on the basis of the total weight of the second alkaline solution.

Still referring to the method of making mesoporous ZSM-5, the dissolution may occur slowly in the presence of a surfactant by gradual heating for at least 4 hours, such as at least 8 hours, at least 12 hours, at least 16 hours, at least 20 hours, or at least 24 hours. Mesopores may be generated using a template mediated technique. Various heating processes or elements are contemplated. For example, the heating may be hydrothermal heating. In one or more embodiments, the heating may occur at a temperature of 50 to 150° C., or a temperature of 90 to 110° C. Furthermore, the duration of heating may range from 30 minutes to 48 hours.

Various second alkaline solutions are contemplated for the dissolution. Suitable second alkaline solutions for use in the dissolving step may include those with a pH of from 0.1 to 0.6, such as from 0.1 to 0.2, from 0.2 to 0.3, from 0.3 to 0.4, from 0.4 to 0.5, from 0.5 to 0.6, or any combination of two or more of these ranges. In some specific embodiments, the second alkaline solution may be an aqueous solution of 0.3 M to 0.5 M NaOH. The temperature of the second alkaline solution may be from 50° C. to 100° C., such as from 50° C. to 60° C., from 60° C. to 70° C., from 70° C. to 80° C., from 80° C. to 90° C., from 90° C. to 100° C., or any combination of two or more of these ranges. Additionally, the dissolving step may occur under stirring, in order to further speed dissolution.

In one embodiment, the second alkaline solution may comprise NaOH. In specific embodiments, the alkaline solution may comprise 0.01 to 0.6 M NaOH, such as from 0.01 to 0.05 M NaOH, from 0.05 to 0.1 M NaOH, from 0.1 to 0.2 M NaOH, from 0.2 to 0.3 M NaOH, from 0.3 to 0.4 M NaOH, from 0.4 to 0.5 M NaOH, from 0.5 M NaOH to 0.6 M NaOH, or any combination of two or more of these ranges.

The second alkaline solution may comprise at least 95 wt. %, such as at least 98 wt. %, at least 99 wt. %, at least 99.9 wt. %, or at least 99.99 wt. % of the surfactant, the base, and water, on the basis of the total weight of the second alkaline solution.

Once formed, the dissolved ZSM-5 solution may then be condensed to yield a solid ZSM-5. Condensing the dissolved ZSM-5 solution may comprise adjusting the pH of the dissolved ZSM-5 solution and subsequently, aging the pH adjusted dissolved ZSM-5 solution.

Still referring to the fabrication of mesoporous ZSM-5, the surfactant assisted modification method may comprise adjusting the pH of the dissolved ZSM-5 solution. The adjustment of the pH is performed by an acidic solution. Various acids are contemplated. In one embodiment, the acidic solution comprises sulfuric acid. In one specific embodiment, the acid is 2 Normality (N) (equivalents/liter) dilute sulfuric acid. In specific embodiments, the pH is adjusted to less than 10, such as to less than 9, from 8 to 10, from 8 to 9, 9 to 10, 8.5 to 9.5, 8.75 to 9.25, or any combination of two or more of these ranges.

Once the pH is adjusted, the pH adjusted dissolved ZSM-5 solution may be aged. The aging, sometimes referred to as hydrothermal aging, may involve maintaining the pH adjusted dissolved ZSM-5 solution at a temperature of 75 to 125° C. for a duration of 12 to 48 hours. For example, the pH adjusted dissolved zeolite solution may be aged at from 75 to 85° C., from 85 to 95° C., from 95 to 105° C., from 105 to 115° C., from 115 to 125° C., or any combination of two or more of these ranges for a duration of at least 4 hours, such as at least 8 hours, at least 12 hours, at least 16 hours, at least 20 hours, at least 24 hours, from 4 hours to 8 hours, from 8 hours to 12 hours, from 12 hours to 16 hours, from 16 hours to 20 hours, from 20 hours to 24 hours, from 24 hours to 28 hours, from 28 hours to 32 hours, from 32 hours to 36 hours, from 36 hours to 40 hours, from 40 hours to 44 hours, from 44 hours to 48 hours, or any combination of two or more of these ranges. Without being limited by theory, it is believed that during aging, the soluble aluminosilicate species may be hydrothermally condensed to form mesophases. The condensing of the dissolved ZSM-5 solution may be performed under still conditions or agitated conditions. Specifically, under still conditions, the dissolved ZSM-5 solution is not stirred or mixed where conversely under agitated conditions the dissolved ZSM-5 solution is mixed or stirred. The solid products formed from condensing the dissolved ZSM-5 solution may be filtered to form a solid ZSM-5, washed thoroughly using distilled water, and then dried. For example, the solid ZSM-5 may be dried for at least 12 hours at a temperature of from 60° C. to 160° C., such as from 60° C. to 80° C., from 80° C. to 100° C., from 100° C. to 120° C., from 120° C. to 140° C., from 140° C. to 160° C., or any combination of two or more of these ranges.

Following drying, the solid ZSM-5 may then be subjected to ion exchange. The ion exchange may occur in the presence of a nitrate solution, e.g., a solution comprising NH₄NO₃. In one or more embodiments, the solid ZSM-5 is ion exchanged thrice with 0.05M NH₄NO₃ solution at 80° C. for 5 hours.

Then, the solid ZSM-5 may be heated to remove the entrained surfactant. For example, the solid zeolite composite may be heated to 500 to 600° C. for 5 to 8 hours, such as from 500 to 520° C., from 520 to 540° C., from 540 to 560° C., from 560 to 580° C., from 580 to 600° C., or any combination of two or more of these ranges, for from 5 hours to 6 hours, from 6 hours to 7 hours, from 7 hours to 8 hours, or any combination of two or more of these ranges. In some specific embodiments, the solid ZSM-5 may be heated to about 570° C. for 7 hours, or 550° C. for 6 hours to vaporize and remove the surfactant. The heating may occur in the presence of an oxidizing atmosphere, such as air. Thus, the mesoporous ZSM-5 may be formed.

Referring now to the zeolite composite catalyst 120 prepared according to method 100 of FIG. 1, a weight ratio of the molybdenum to the one or more active metals in the zeolite composite catalyst 120 may be from 100:0.5 to 100:2, such as from 100:0.5 to 100:0.7, from 100:0.7 to 100:0.9, from 100:0.9 to 100:1.1, from 100:1.1 to 100:1.3, from 100:1.3 to 100:1.5, from 100:1.5 to 100:1.7, from 100:1.7 to 100:1.9, from 100:1.9 to 100:2.0, or any combination of two or more of these ranges.

The zeolite composite catalyst 102 may comprise from 40 wt. % to 50 wt. % of the mesoporous mordenite, on the basis of the total weight of the zeolite composite catalyst. For example, the zeolite composite catalyst 102 may comprise from 40 wt. % to 42 wt. %, from 42 wt. % to 44 wt. %, from 44 wt. % to 46 wt. %, from 46 wt. % to 48 wt. %, from 48 wt. % to 50 wt. %, or any combination of two or more of these ranges of the mesoporous mordenite, on the basis of the total weight of the zeolite composite catalyst 102.

The zeolite composite catalyst 102 may comprise from 14 wt. % to 25 wt. % of the mesoporous ZSM-5, on the basis of the total weight of the zeolite composite catalyst 102. For example, the zeolite composite catalyst 102 may comprise from 14 wt. % to 16 wt. %, from 16 wt. % to 18 wt. %, from 18 wt. % to 20 wt. %, from 20 wt. % to 22 wt. %, from 22 wt. % to 24 wt. %, from 24 wt. % to 25 wt. %, or any combination of two or more of these ranges of the mesoporous ZSM-5, on the basis of the total weight of the zeolite composite catalyst 102.

The ratio of mesoporous mordenite to mesoporous ZSM-5 in the zeolite composite catalyst 102 may be from 2.00:1 to 4.00:1, such as from 2.00:1 to 2.25:1, from 2.25:1 to 2.50:1, from 2.50:1 to 2.75:1, from 2.75:1 to 3.00:1, from 3.00:1 to 3.25:1, from 3.25:1 to 3.5:1, from 3.5:1 to 3.75:1, from 3.75:1 to 4.00:1, or any combination of two or more of these ranges.

The zeolite composite catalyst 102 may comprise from 1 wt. % to 4 wt. % of the molybdenum, on the basis of the total weight of the zeolite composite catalyst 102. For example, the zeolite composite catalyst 102 may comprise from 1 wt. % to 1.5 wt. %, from 1.5 wt. % to 2.0 wt. %, from 2.0 wt. % to 2.5 wt. %, from 2.5 wt. % to 3.0 wt. %, from 3.0 wt. % to 3.5 wt. %, from 3.5 wt. % to 4.0 wt. %, or any combination of two or more of these ranges of the molybdenum, on the basis of the total weight of the zeolite composite catalyst 102.

The zeolite composite catalyst 102 may comprise from 0.05 wt. % to 0.5 wt. % of the one or more active metals, on the basis of the total weight of the zeolite composite catalyst 102. For example, the zeolite composite catalyst 102 may comprise from 0.05 wt. % to 0.1 wt. %, from 0.1 wt. % to 0.2 wt. %, from 0.2 wt. % to 0.3 wt. %, from 0.3 wt. % to 0.4 wt. %, from 0.4 wt. % to 0.5 wt. %, or any combination of two or more of these ranges of the one or more active metals, on the basis of the total weight of the zeolite composite catalyst 120.

The zeolite composite catalyst 120 may comprise from 10 wt. % to 30 wt. % wt. % of the binder, on the basis of the total weight of the zeolite composite catalyst 102. For example, the zeolite composite catalyst 120 may comprise from 10 wt. % to 14 wt. %, from 14 wt. % to 18 wt. %, from 18 wt. % to 22 wt. %, from 22 wt. % to 26 wt. %, from 26 wt. % to 30 wt. %, or any combination of two or more of these ranges, of the binder.

The zeolite composite catalyst 120 may comprise a majority of the combined weight of the mesoporous ZSM-5, the mesoporous mordenite, the molybdenum, the one or more active metals, and optional binder. For example, the zeolite composite catalyst 102 may comprise at least 50 wt. %, at least 75 wt. %, at least 85 wt. %, at least 95 wt. %, at least 95 wt. %, at least 98 wt. %, at least 99 wt. %, at least 99.9 wt. %, at least 99.99 wt. %, at least 99.999 wt. %, or even at least 99.9999 wt. % of the combined weight of the mesoporous ZSM-5, the mesoporous mordenite, the molybdenum, the one or more active metals, and optional binder, on the basis of the total weight of the zeolite composite catalyst 120.

From a property standpoint, the molar ratio of silica to aluminum in the zeolite composite catalyst 102 may be from 18 to 500. For example, the molar ratio of silica to aluminum in the zeolite composite catalyst 120 may be from 18 to 20, from 20 to 25, from 25 to 30, from 30 to 50, from 50 to 75, from 75 to 100, from 100 to 125, from 125 to 150, from 150 to 200, from 200 to 250, from 250 to 300, from 300 to 350, from 350 to 400, from 400 to 450, from 450 to 500, or an combination of two or more of these ranges.

The zeolite composite catalyst 120 may have a surface area defined by a Brunauer-Emmett-Teller (BET) analysis (also known as the Specific Surface Area) of at least 600 m²/g, or a BET surface area of at least 700 m²/g. Further, the zeolite composite catalyst 120 may have an external surface area of at least 300 m²/g, of at least 350 m²/g, of at least 400 m²/g, or 400 to 700 m²/g.

In one or more embodiments, the zeolite composite catalyst 120 may have a total pore volume of 0.20 to 3.0 cubic centimeters per gram (cm³/g), or 0.30 to 1.0 cm³/g. Moreover, the zeolite composite catalyst 120 may have a total pore volume of at least 0.30 cm³/g, or of at least 0.40 cm³/g, or at least 0.50 cm³/g. Total pore volume is determined according to the BET analysis.

In some embodiments, the zeolite composite catalyst 120 may have an average pore diameter of at least 2 nm, at least 3 nm, at least 4 nm, at least 5 nm, or at least 6 nm. In some embodiments, the zeolite composite catalyst 120 may have an average pore diameter of from 2 nm to 10 nm, such as from 2 nm to 3 nm, from 3 nm to 4 nm, from 4 nm to 5 nm, from 5 nm to 6 nm, from 7 nm to 8 nm, from 8 nm to 9 nm, from 9 nm to 10 nm, or any combination of two or more of these ranges. Average pore volume is determined according to the BET analysis.

Referring now to the embodiments of FIG. 2, a method of converting C₉₊ alkyl aromatic hydrocarbons to a product stream comprising benzene, toluene, and xylene is depicted in the context of a system for converting C₉₊ alkyl aromatic hydrocarbons to a product stream comprising benzene, toluene, and xylene. The system and method described in FIG. 2 represents only one use case for the presently described catalyst, and should not be considered as limiting on the methods described herein for making such catalysts.

The starting material for the method may be a feed stream 510 comprising C₉₊ alkylaromatic hydrocarbons. In some embodiments, the feed stream 510 may be a heavy reformate stream, or a portion thereof. In some embodiments, the feed stream 510 may comprise at least 50 wt. %, at least 60 wt. %, at least 70 wt. %, at least 80 wt. %, at least 90 wt. %, at least 95 wt. %, or even at least 99 wt. % of the C₉₊ alkylaromatic hydrocarbons.

The method may comprise heating the feed stream 510 comprising C₉₊ alkylaromatic hydrocarbons from a feed source 505 with a heater unit 511. As shown, the heater unit 511 may be a heat exchanger which receives a heated stream 508, for example, a heated water stream to heat the feed stream 510 prior to delivery to a reactor 513. The heated stream 508 may also be the effluent of the reaction zone. Other methods of heating the feed are contemplated.

The reactor system may include a reactor 513 with zeolite composite catalyst used in transalkylation catalyst zone 515 as shown in FIG. 2, or may include multiple reactors 513 or stages. The reactor 513 is depicted as a down flow 514 reactor but that is one of many possibilities. Similarly, in the embodiment of FIG. 2, the reactor 513 has a fixed cylindrical bed of catalyst; however, other reaction configurations utilizing moving beds of catalyst, radial-flow reactors, or fluidized beds may be employed. Prior to the feed being delivered, the zeolite composite catalyst in transalkylation catalyst zone 515 may be reduced, for example, reduced with hydrogen gas 512. In one embodiment, the zeolite composite catalyst is reduced by contacting the zeolite composite catalyst with hydrogen gas 512 at a temperature of 400° C. to 500° C., such as from 425° C. to 450° C., from 450° C. to 475° C., from 475° C. to 500° C., or any combination of two or more of these ranges.

Referring still to FIG. 2, the feed stream 510 contacts the reduced composite zeolite catalyst and hydrogen 512 in the transalkylation catalyst zone 515 of the reactor 513 to produce a transalkylation product. Specifically, the feed stream 510 is transalkylated in the vapor phase and in the presence of hydrogen 512. The hydrogen 512 may be delivered with the feed stream 510 in an amount from 0.1 to 10 moles of hydrogen per mole of alkylaromatics. This ratio of hydrogen to alkylaromatics is also referred to as the hydrogen-to-hydrocarbon ratio. The transalkylation results in the production of a transalkylation effluent stream 516 comprising product hydrocarbons, specifically, hydrocarbons having mixed xylene content, as well as unconverted feed, toluene, and benzene. Various conditions are contemplated for the reactor 513. Specifically, the feed may contact the reduced zeolite composite catalyst in the transalkylation catalyst zone 515 at a temperature between 50° C. and 600° C., such as 50° C. to 75° C., 75° C. to 100° C., 100° C. to 150° C., 150° C. to 200° C., 200° C. to 250° C., 250° C. to 300° C., 300° C. to 350° C., 350° C. to 400° C., 400° C. to 450° C., 450° C. to 500° C., 500° C. to 550° C., 550° C. to 600° C., or any combination of two or more of these ranges; a pressure of from 0.5 mega Pascal (MPa) to 15.0 MPa, such as from 0.5 MPa to 1.0 MPa, from 1.0 MPa to 2.0 MPa, from 2.0 MPa to 3.0 MPa, from 3.0 MPa to 5.0 MPa, from 5.0 MPa to 7.5 MPa, from 7.5 MPa to 10.0 MPa, from 10.0 MPa to 12.5 MPa, from 12.5 MPa to 15 MPa, or any combination of two or more of these ranges; a liquid hourly space velocity (LHSV) is in the range of 1.0 hr⁻¹ to 5.0 hr⁻¹, such as from 1.0 hr⁻¹ to 2.0 hr⁻¹ from 2.0 hr⁻¹ to 3.0 hr⁻¹, from 3.0 hr⁻¹ to 4.0 hr⁻¹, from 4.0 hr⁻¹ to 5.0 hr⁻¹, or any combination of two or more of these ranges; and a hydrogen to hydrocarbon weight ratio of from 1 to 4, such as from 1 to 2, from 2 to 3, from 3 to 4, or any combination of two or more of these ranges.

The transalkylation effluent stream 516 may be cooled using a cooler 517. The cooler 517 may be a heat exchanger, condenser, or any other suitable cooling device. As shown, the cooler 517 is a heat exchanger which includes a cooling stream 518. Next, the transalkylation effluent stream 516 may be fed to a stripper column 520, where C₁-C₅ and lighter hydrocarbons 522 are separated from the transalkylation effluent stream 516.

Still referring to FIG. 2, the product 524 of the stripper column 520, which may be discharged from the bottom of the stripper column 520, may include a light recovery stream 526 comprising benzene and toluene, a mixed C₈ aromatics product 528, and a heavy recycle stream 530. These all may subsequently be separated in one or more reaction vessels 525, 527, 529. The mixed C₈ aromatics product 528 can be sent for recovery of p-xylene 532 and other valuable isomers 534. The light recovery stream 526 may undergo benzene and toluene recovery to form a benzene and toluene stream 536 with the remainder portion recycled as a light recycle stream 538 to the transalkylation zone or the feed source 505. The heavy recycle stream 530 may contain substantially all of the C₉ and heavier aromatics and may be partially or totally recycled to the transalkylation reaction zone, or delivered to the feed source 505 for recycle, or removed from the process for disposal or other processing.

Numerous aspects are presently disclosed herein, including Aspects 1-20.
Aspect 1: A method of producing a zeolite composite catalyst, the method comprising: impregnating a zeolite composite with molybdenum to form a molybdenum impregnated zeolite composite, wherein the zeolite composite comprises a mesoporous mordenite and a mesoporous ZSM-5; calcining the molybdenum impregnated zeolite composite to form a calcined, molybdenum impregnated zeolite composite; impregnating the calcined, molybdenum impregnated zeolite composite with one or more active metals selected from platinum, ruthenium, nickel, tungsten, chromium, cerium, or gold, to yield a multi-metallic impregnated zeolite composite; and calcining the multi-metallic impregnated zeolite composite to produce the zeolite composite catalyst.
Aspect 2: The method of aspect 1, wherein the one or more active metals are selected from platinum, nickel, ruthenium, or combinations thereof.
Aspect 3: The method of aspects 1-2, wherein a ratio of the molybdenum to the one or more active metals is from 6:1 to 80:1.
Aspect 4: The method of aspects 1-3, wherein the zeolite composite catalyst has a ratio of a ratio of mesoporous mordenite to mesoporous ZSM-5 of from 1:1 to 5:1 by weight.
Aspect 5: The method of aspects 1-4, wherein the zeolite composite catalyst comprises: from 40 to 50 wt. % of the mesoporous mordenite; from 14 to 25 wt. % of the mesoporous ZSM-5; from 1 to 4 wt. % of the molybdenum; and from 0.05 to 0.5 wt. % of the one or more active metals.
Aspect 6: The method of aspects 1-5, wherein the zeolite composite catalyst further comprise a binder, and wherein the zeolite composite catalyst comprises at least 99 wt. % of the combined weight of the mesoporous ZSM-5, the mesoporous mordenite, the molybdenum, the one or more active metals, and the binder, on the basis of the total weight of the zeolite composite catalyst.
Aspect 7: The method of aspects 1-6, wherein the zeolite composite catalyst has an average pore diameter of 2 nm to 10 nm.
Aspect 8: The method of aspects 1-7, wherein the zeolite composite further comprises an alumina binder.
Aspect 9: The method of aspects 1-8, wherein the mesoporous mordenite has a Si/Al molar ratio of from 20 to 300, and wherein the mesoporous ZSM-5 has a Si/Al molar ratio of from 5 to 500.
Aspect 10: The method of aspects 1-9, wherein: the mesoporous mordenite has a mesopore volume of from 0.2 to 3.0 cm³/g and a micropore volume of from 0.2 to 3.0 cm³/g; and the mesoporous ZSM-5 has a mesopore volume of from 0.2 to 3.0 cm³/g and a micropore volume of from 0.2 to 3.0 cm³/g.
Aspect 11: The method of aspects 1-10, further comprising forming the zeolite composite by a process comprising mixing the mesoporous ZSM-5, the mesoporous mordenite, and an alumina binder.
Aspect 12: The method of aspects 1-11, further comprising: forming the mesoporous mordenite by a process comprising: dissolving, in a first alkaline solution comprising a first surfactant, a microporous mordenite while heating, stirring, or both to yield a dissolved mordenite solution; condensing the dissolved mordenite solution to yield a solid mordenite from the dissolved mordenite solution, wherein condensing the dissolved mordenite solution comprises: adjusting a pH of the dissolved mordenite solution; and aging the pH adjusted dissolved mordenite solution; and heating the solid mordenite to remove the first surfactant and form the mesoporous mordenite; and forming the mesoporous ZSM-5 by a process comprising: dissolving, in a second alkaline solution comprising a second surfactant, a microporous ZSM-5 while heating, stirring, or both to yield a dissolved ZSM-5 solution; condensing the dissolved ZSM-5 solution to yield a solid ZSM-5 from the dissolved ZSM-5 solution, wherein condensing the dissolved ZSM-5 solution comprises: adjusting a pH of the dissolved ZSM-5 solution; and aging the pH adjusted dissolved ZSM-5 solution; and heating the solid ZSM-5 to remove the second surfactant and form the mesoporous ZSM-5.
Aspect 13: The method of aspect 12, wherein: the first surfactant and the second surfactant are each cetyltrimethyl ammonium bromide; and the dissolved ZSM-5 solution and the dissolved mordenite solution each comprise from 1 to 8 wt. % of the cetyltrimethyl ammonium bromide.
Aspect 14: The method of aspects 12-13, wherein the first alkaline solution and the second alkaline solution are each aqueous 0.1 M to 0.6 M NaOH solutions.
Aspect 15: The method of aspects 12-14, wherein the adjusting the pH of the dissolved ZSM-5 solution, the adjusting the pH of the dissolved mordenite solution, or both comprises adding an acid to each respective solution such that the pH of the respective pH adjusted solution is less than 10.
Aspect 16: The method of aspects 12-15, wherein the aging comprises maintaining the pH adjusted dissolved ZSM-5 solution, pH adjusted dissolved mordenite solution, or both at a temperature of 75 to 125° C. for a duration of 12 to 48 hours.
Aspect 17: The method of aspects 12-16, wherein the first alkaline solution and the second alkaline solution are both 0.35 M to 0.45 M NaOH solutions.
Aspect 18: A zeolite composite catalyst produced according to the method of aspects 1-17.
Aspect 19. A method of converting C₉₊ alkyl aromatic hydrocarbons to a product stream comprising benzene, toluene, and xylene, the method comprising: reducing the zeolite composite catalyst of aspect 18 by contacting the zeolite composite catalyst with hydrogen gas at 400° C. to 500° C.; contacting a feed comprising C₉₊ alkylaromatic hydrocarbons with the reduced zeolite composite catalyst and hydrogen in a transalkylation zone of a reactor to produce a transalkylation product; and collecting at least the xylenes from the transalkylation product.
Aspect 20: The method of aspect 19, wherein the feed contacts the reduced zeolite composite catalyst at a pressure between 1.0 MPa to 3.0 MPa, a temperature of 200° C. to 500° C., a space velocity of 1.0 h⁻¹ to 5.0 h⁻¹, and a hydrogen to hydrocarbon ratio of 1 to 4.

### EXAMPLES

The described embodiments will be further clarified by the following examples and comparative examples. The catalysts described in these examples are exemplary embodiments only, and are not intended to limit the general description of the zeolite composite catalyst.

The synthesis of Comparative Catalyst A, which includes only a single metal, is described in Comparative Example A. The synthesis of Catalysts B, D, E, F, and H, which all include sequential addition of the molybdenum and the one or more active metals is described in Example 2. The synthesis of Comparative Catalysts C and G, which include co-addition of the molybdenum and the one or more active metals is described in Example 3. The composition and loading method of each catalyst are described in Table 1. The performance of Comparative Catalysts A, C, and G, and Catalysts B, D, E, F, and H was then evaluated to determine their function for transkalyation of C₉₊ alkyl aromatic hydrocarbons, as described in Example 4.

In each example, the catalyst composition comprises 48.24 wt. % mordenite, 16.08 wt. % ZSM-5, and 4 wt. % of molybdenum. The binder is an alumina binder (Cataloid, AP-3, obtained from Catalysts & Chemicals Industries Co., Ltd (CCIC), Japan),

TABLE 1

| Catalyst | Loading Method | Catalyst Composition |
|---|---|---|
| Comparative | Single metal | Mordenite (48.24%) |
| Catalyst-A | | ZSM-5 (16.08%) |
| | | Binder (31.68%) |
| | | Mo (4%) |
| Catalyst-B | Sequential | Mordenite (48.24%) |
| | addition | ZSM-5 (16.08%) |
| | | Binder (31.63%) |
| | | Mo (4%) |
| | | Pt (0.03%) |
| Comparative | Co-Addition | Mordenite (48.24%) |
| Catalyst-C | | ZSM-5 (16.08%) |
| | | Binder (31.65%) |
| | | Mo (4%) |
| | | Pt (0.03%) |
| Catalyst-D | Sequential | Mordenite (48.24%) |
| | addition | ZSM-5 (16.08%) |
| | | Binder (31.38%) |
| | | Mo (4%) |
| | | Ni (0.3%) |
| Catalyst-E | Sequential | Mordenite (48.24%) |
| | addition | ZSM-5 (16.08%) |
| | | Binder (31.18%) |
| | | Mo (4%) |
| | | Ru (0.05%) |
| Catalyst-F | Sequential | Mordenite (48.24%) |
| | addition | ZSM-5 (16.08%) |
| | | Binder (31.18%) |
| | | Mo (4%) |
| | | Ni (0.5%) |
| Comparative | co-addition | Mordenite (48.24%) |
| Catalyst-G | | ZSM-5 (16.08%) |
| | | Binder (31.63%) |
| | | Mo (4%) |
| | | Pt (0.05%) |
| Catalyst-H | sequential | Mordenite (48.24%) |
| | addition | ZSM-5 (16.08%) |
| | | Binder (31.63%) |
| | | Mo (4%) |
| | | Pt (0.05%) |

### Comparative Example A

### Mesoporous Mordenite Production

Microporous mordenite (Si/Al molar ratio=30), was disintegrated using an aqueous 0.40 M NaOH solution by gradual heating (without stirring) at 100° C. for 24 hours (h). The heating was carried out in the presence of CTAB (4.45 wt. %). The mixture was cooled down and then the pH was adjusted to 9.0 through the addition of dilute sulfuric acid (2N). The mixture was stirred for 24 hours (h) and then aged at 100° C. for 24 h to form a solid mordenite. The solid mordenite was filtered, washed thoroughly using distilled water, dried at 80° C. overnight, then calcined at 570° C. for 7 h to remove the surfactant (CTAB). The solid composite material thus obtained was ion-exchanged three times with 0.05 M NH₄NO₃ solution at 80° C. for 5 h, to produce the mesoporous mordenite.

TABLE 2
Mesoporous Mordenite Physical Properties

| | Silica to | | | | | |
|---|---|---|---|---|---|---|
| | Alumina | S/BET | S/Micro | S/Meso | V/Micro | V/Meso |
| | Ratio | (m²/g) | (CC/g) | (CC/g) | (cm³/g) | (cm³/g) |
| Mordenite | 20 | 451 | 425 | 26 | 0.18 | 0.03 |
| Mesoporous | 38 | 546 | 192 | 353 | 0.17 | 0.32 |
| Mordenite | | | | | | |

### Mesoporous ZSM-5 Production

Microporous ZSM-5 (Si/Al molar ratio=40), was disintegrated using 0.40 M NaOH solution by gradual heating (without stirring) at 100° C. for 24 hours (h). The heating was carried out in the presence of CTAB (4.45 wt. %). The mixture was cooled down and then the pH was adjusted to 9.0 through the addition of dilute sulfuric acid (2N). The mixture was then stirred for 24 hours (h) and then aged at 100° C. for 24 h to form a solid zeolite composite. The solid zeolite composite was filtered, washed thoroughly using distilled water, dried at 80° C. overnight, then calcined in air at 570° C. for 7 h to remove the surfactant (CTAB). The solid composite material thus obtained was ion-exchanged three times with 0.05 M NH₄NO₃ solution at 80° C. for 5 h to form the mesoporous ZSM-5.

TABLE 3
Mesoporous ZSM-5 Physical Properties

| | Silica to | | | | | |
|---|---|---|---|---|---|---|
| | Alumina | S/BET | S/Micro | S/Meso | V/Micro | V/Meso |
| | Ratio | (m²/g) | (CC/g) | (CC/g) | (cm³/g) | (cm³/g) |
| ZSM-5 | 28 | 372 | 332 | 40 | 0.16 | 0.06 |
| Mesoporous | 32 | 512 | 252 | 260 | 0.16 | 0.24 |
| ZSM-5 | | | | | | |

### Catalyst Formation

The resulting mesoporous mordenite and mesoporous ZSM-5 were physically mixed in a 3:1 weight ratio with untreated ZSM and made into extrudates by mixing about 67 wt. % of the combined weight of mesoporous mordenite and mesoporous ZSM-5 with about 33 wt. % of an alumina binder (Cataloid AP-3), on the basis of the total weight of mesoporous ZSM-5, mesoporous mordenite, and binder. Then, the extrudate was loaded with 4 wt. % of molybdenum in the form of ammonium molybdate tetrahydrate through wet impregnation. Then, the impregnated solid zeolite was calcined in air at 450° C. for 5 h. The resulting composite is designated as Comparative Catalyst A in Table 1.

### Example 2 (Formation of Catalysts B, D, E, F, and H by Sequential Addition)

Comparative Catalyst A was further loaded with one or more active metals through a wet impregnation step, in the amounts and types indicated in Table 1. Specifically, the material of Comparative Catalyst A was loaded Pt in the form of ammonium tetrachloroplatinate, Ni in the form of nickel (II) nitrate hexahydrate, or Ru in the form of ruthenium chloride. After impregnation with their respective additional metals, the materials of catalysts B, D, E, F, and H were calcined in air at 450° C. for 5 h a second time.

### Example 3 (Formation of Comparative Catalysts C and G by Co-Addition)

An extrudate comprising the mesoporous mordenite, mesoporous ZSM-5, and binder was produced as in Example 1. Then, the extrudate was loaded with 4 wt. % of molybdenum in the form of ammonium molybdate tetrahydrate and varying amounts of platinum in the form of ammonium tetrachloroplatinate, in a single wet impregnation step. After impregnation, the materials of catalysts C and G were calcined in air at 450° C. for 5 h.

### Example 4 (Activity Studies)

Activity studies for the catalysts described in Table 1 were performed on a bench top, fixed-bed reactor using industrial reformate feedstock diluted with toluene (having a composition described in Table 4). First, the catalysts were activated and in-situ reduced under 100 m¹/min flow of pure hydrogen gas at 450° C. and were kept at this temperature for 3 hours. Then, the pressure of the reactor was increased to 20 bar and held for another 5 hours. The catalytic tests were performed under the following conditions: 350° C. temperature, 20 bar total pressure, hydrogen/hydrocarbon molar ratio of 4, reaction time of 12 days, and WHSV=4.2/h. The amount of catalyst (extrude size 0.2-0.4 mm) in each fixed-bed was 2 g diluted with SiC to a total bed volume of 20 ml. The listed activity and yields were sustained over 300 hours of operation at 350° C. for 300 hours without loss of activity. Table 5 provides a summary of the results.

TABLE 4
Feed-in (wt. %)

| | |
|---|---|
| Toluene-in | 40.6 |
| 1,3-methyl ethylbenzene-in | 6.08 |
| 1,4-methyl ethylbenzene-in | 3.33 |
| 1,2-methyl ethylbenzene-in | 2.34 |
| MEB-in (wt. %) | 11.7 |
| 1,3,5-trimethylbenzene-in | 7.99 |
| 1,2,4-trimethylbenzene-in | 24.24 |
| 1,2,3-trimethylbenzene-in | 4.22 |
| TMB-in (wt. %) | 36.5 |
| C10 + Heavies | 11.2 |

TABLE 5

| | | | Avg. | Avg. | | |
|---|---|---|---|---|---|---|
| | | | Total | Toluene | Avg. | |
| | Ave. MEB | Avg. TMB | Xylenes | in the | benzene | |
| | Conversion | Conversion | yield | product | yield | |
| Catalyst ID | (wt. %) | (wt. %) | (wt. %) | (wt. %) | (wt. %) | EB |
| CC-A | 79 | 45 | 35.9 | 26.7 | 4.7 | 2.45 |
| Catalyst-B | 88 | 45 | 36.4 | 26.4 | 4.8 | 1.39 |
| CC-C | 87 | 46 | 36.76 | 26.5 | 4.9 | 1.52 |
| Catalyst-D | 81 | 45 | 35.96 | 26.5 | 4.7 | 2.10 |
| Catalyst-E | 87 | 46 | 36.83 | 26.9 | 5 | 1.56 |
| Catalyst-F | 84 | 45 | 36.37 | 26.2 | 4.8 | 1.90 |
| CC-G | 84.63 | 47.36 | 36.34 | 26.4 | 4.75 | 1.78 |
| Catalyst-H | 89.08 | 48.45 | 36.56 | 27.7 | 4.62 | 1.32 |

CC-A (mono-metallic (Mo)) exhibited good overall catalyst performance at 350° C. with methyl-ethyl benzene (MEB) and triethyl benzene (TMB) conversions of 79% and 45%, respectively, and xylene yields of 35.9%. However, the ethylbenzene (EB) content in the C₈₊ aromatic stream is 2.45 wt. % which is high enough to cause inefficiencies in the paraxylene recovery unit. The addition of second metals (Pt, Ru or Ni) to Comparative Catalyst-A (as illustrated in Catalysts B-F) reduced the EB content in the C₈₊ aromatics stream while simultaneously increasing the xylene yields and MEB conversions. The higher dealkylation capacity of the multi-metal catalysts and lower EB formation results in an improved yield to the desired xylenes fraction (average of ~36.8) and higher conversion of MEB around 87%.

Additionally, the sequential addition, rather than co-addition, of the second metal was found to decrease EB yield further. Otherwise identical pairs of catalysts (e.g., G&H and B&C) show the improvement in EB yield from sequential addition of the second metal. For example, Catalyst C uses co-addition of Mo and Pt and has an EB yield of 1.52 wt. %, whereas Catalyst B (otherwise identical to Catalyst C) uses sequential addition and has an EB yield of only 1.39 wt. %, an 8.55% reduction in EB yield. Similarly Catalyst G uses co-addition of Mo and Pt and has an EB yield of 1.78 wt. %, whereas Catalyst H uses sequential addition (otherwise identical to Catalyst G) has an EB yield of only 1.32 wt. %, a 25.8% reduction in EB yield.

What is claimed is:

1. A method of producing a zeolite composite catalyst, the method comprising:
impregnating a zeolite composite with molybdenum to form a molybdenum impregnated zeolite composite, wherein the zeolite composite comprises a mesoporous mordenite and a mesoporous ZSM-5;
calcining the molybdenum impregnated zeolite composite to form a calcined, molybdenum impregnated zeolite composite;
impregnating the calcined, molybdenum impregnated zeolite composite with one or more active metals selected from platinum, ruthenium, nickel, tungsten, chromium, cerium, or gold, to yield a multi-metallic impregnated zeolite composite; and
calcining the multi-metallic impregnated zeolite composite to produce the zeolite composite catalyst.

2. The method of claim 1, wherein the one or more active metals are selected from platinum, nickel, ruthenium, or combinations thereof.

3. The method of claim 1, wherein a ratio of the molybdenum to the one or more active metals is from 6:1 to 80:1.

4. The method of claim 1, wherein the zeolite composite catalyst has a ratio of a ratio of mesoporous mordenite to mesoporous ZSM-5 of from 1:1 to 5:1 by weight.

5. The method of claim 1, wherein the zeolite composite catalyst comprises:
from 40 to 50 wt. % of the mesoporous mordenite;
from 14 to 25 wt. % of the mesoporous ZSM-5;
from 1 to 4 wt. % of the molybdenum; and
from 0.05 to 0.5 wt. % of the one or more active metals.

6. The method of claim 1, wherein the zeolite composite catalyst further comprise a binder, and wherein the zeolite composite catalyst comprises at least 99 wt. % of the combined weight of the mesoporous ZSM-5, the mesoporous mordenite, the molybdenum, the one or more active metals, and the binder, on the basis of the total weight of the zeolite composite catalyst.

7. The method of claim 1, wherein the zeolite composite catalyst has an average pore diameter of 2 nm to 10 nm.

8. The method of claim 1, wherein the zeolite composite further comprises an alumina binder.

9. The method of claim 1, wherein the mesoporous mordenite has a Si/Al molar ratio of from 20 to 300, and wherein the mesoporous ZSM-5 has a Si/Al molar ratio of from 5 to 500.

10. The method of claim 1, wherein:
the mesoporous mordenite has a mesopore volume of from 0.2 to 3.0 cm³/g and a micropore volume of from 0.2 to 3.0 cm³/g; and
the mesoporous ZSM-5 has a mesopore volume of from 0.2 to 3.0 cm³/g and a micropore volume of from 0.2 to 3.0 cm³/g.

11. The method of claim 1, further comprising forming the zeolite composite by a process comprising mixing the mesoporous ZSM-5, the mesoporous mordenite, and an alumina binder.

12. The method of claim 1, further comprising:
forming the mesoporous mordenite by a process comprising:
dissolving, in a first alkaline solution comprising a first surfactant, a microporous mordenite while heating, stirring, or both to yield a dissolved mordenite solution;
condensing the dissolved mordenite solution to yield a solid mordenite from the dissolved mordenite solution, wherein condensing the dissolved mordenite solution comprises:
adjusting a pH of the dissolved mordenite solution; and
aging the pH adjusted dissolved mordenite solution; and
heating the solid mordenite to remove the first surfactant and form the mesoporous mordenite; and
forming the mesoporous ZSM-5 by a process comprising:
dissolving, in a second alkaline solution comprising a second surfactant, a microporous ZSM-5 while heating, stirring, or both to yield a dissolved ZSM-5 solution;
condensing the dissolved ZSM-5 solution to yield a solid ZSM-5 from the dissolved ZSM-5 solution, wherein condensing the dissolved ZSM-5 solution comprises:
adjusting a pH of the dissolved ZSM-5 solution; and
aging the pH adjusted dissolved ZSM-5 solution; and
heating the solid ZSM-5 to remove the second surfactant and form the mesoporous ZSM-5.

13. The method of claim 12 wherein:
the first surfactant and the second surfactant are each cetyltrimethyl ammonium bromide; and
the dissolved ZSM-5 solution and the dissolved mordenite solution each comprise from 1 to 8 wt. % of the cetyltrimethyl ammonium bromide.

14. The method of claim 12, wherein the first alkaline solution and the second alkaline solution are each aqueous 0.1 M to 0.6 M NaOH solutions.

15. The method of claim 12, wherein the adjusting the pH of the dissolved ZSM-5 solution, the adjusting the pH of the dissolved mordenite solution, or both comprises adding an acid to each respective solution such that the pH of the respective pH adjusted solution is less than 10.

16. The method of claim 12, wherein the aging comprises maintaining the pH adjusted dissolved ZSM-5 solution, pH adjusted dissolved mordenite solution, or both at a temperature of 75 to 125° C. for a duration of 12 to 48 hours.

17. The method of claim 12 wherein the first alkaline solution and the second alkaline solution are both 0.35 M to 0.45 M NaOH solutions.

18. A zeolite composite catalyst produced according to the method of claim 1.

19. A method of converting C₉₊ alkyl aromatic hydrocarbons to a product stream comprising benzene, toluene, and xylene, the method comprising:
reducing the zeolite composite catalyst of claim 18 by contacting the zeolite composite catalyst with hydrogen gas at 400° C. to 500° C.;
contacting a feed comprising C₉₊ alkylaromatic hydrocarbons with the reduced zeolite composite catalyst and hydrogen in a transalkylation zone of a reactor to produce a transalkylation product; and
collecting at least the xylenes from the transalkylation product.

20. The method of claim 19, wherein the feed contacts the reduced zeolite composite catalyst at a pressure between 1.0 MPa to 3.0 MPa, a temperature of 200° C. to 500° C., a space velocity of 1.0 h⁻¹ to 5.0 h⁻¹, and a hydrogen to hydrocarbon ratio of 1 to 4.
